(19) United States
(12) Patent Application Publication
(10) Pub. No.: US 2021/0199615 A1
(43) Pub. Date: Jul. 1, 2021

(54) ELECTROCHEMICAL-BASED ANALYTICAL TEST STRIP WITH ELECTRODE VOLTAGE SENSING CONNECTIONS AND HAND-HELD TEST METER FOR USE THEREWITH

(71) Applicant: Lifescan IP Holdings, LLC, Malvern, PA (US)

(72) Inventors: David Elder, Malvern, PA (US); David McColl, Malvern, PA (US)

(21) Appl. No.: 17/201,534

(22) Filed: Mar. 15, 2021

Related U.S. Application Data
(63) Continuation of application No. 16/066,133, filed on Jun. 26, 2018.
(60) Provisional application No. 62/271,473, filed on Dec. 28, 2015.

(51) Int. Cl. G01N 27/327

(52) U.S. Cl. CPC G01N 27/3273; G01N 27/3272

(57) ABSTRACT

A hand-held test meter for use with an electro-chemical-based analytical test strip in the determination of an analyte in a bodily fluid sample includes a housing; a strip port connector disposed at least partially within the housing and configured to receive an electro-chemical based analytical test strip; a micro-controller disposed in the housing and configured to generate a micro-controller command signal; an electrode bias drive circuit block disposed in the housing and configured to generate a bias drive signal based on the micro-controller command signal, and a dynamic bias drive adjustment circuit block disposed in the housing and configured to receive at least one sensed electrode voltage and to adjust a bias drive signal from the electrode bias drive circuit block based on the sensed electrode voltage to create an adjusted bias drive signal.

### CROSS REFERENCE TO RELATED APPLICATION(S)

This application is a divisional of U.S. patent application Ser. No. 16/066,133, filed Jun. 26, 2018, which is a national stage application under 35 U.S.C. § 371 of International Application No. PCT/EP2016/082357, filed Dec. 22, 2016, which claims priority under applicable portions of 35 U.S.C. § 119 to U.S. Patent Application Ser. No. 62/271,473, filed Dec. 28, 2015, the entire contents of each application being incorporated herein by reference.

### FIELD OF THE INVENTION

The present invention relates, in general, to medical devices and, in particular, to electrochemical-based analytical test strips and hand-held test meters for use therewith.

### DESCRIPTION OF RELATED ART

The determination (e.g., detection and/or concentration measurement) of an analyte in, or a characteristic of, a fluid sample is of particular interest in the medical field. For example, it can be desirable to determine glucose, ketone bodies, cholesterol, lipoproteins, triglycerides, acetaminophen, hematocrit and/or HbA1c concentrations in a sample of a bodily fluid such as urine, blood, plasma or interstitial fluid. Such determinations can be achieved using analytical test strips, based on, for example, visual, photometric or electrochemical techniques in conjunction with a hand-held test meter. Conventional electrochemical-based analytical test strips are described in, for example, U.S. Pat. Nos. 5,708,247 and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified exploded perspective view of an electrochemical-based analytical test strip according to an embodiment of the present invention;

FIG. 2 is a simplified top view of a patterned conductor layer and substrate layer of the electrochemical-based analytical test strip of FIG. 1 with the location of an enzymatic reagent layer depicted by dashed lines;

FIG. 3 is a simplified top view of a hand-held test meter according to an embodiment of the present invention;

FIG. 4 is a simplified block diagram of the hand-held test meter of FIG. 4; and

FIG. 5 is a simplified schematic diagram of an automatic bias drive adjustment circuit block for a single working electrode (WE) and reference electrode (REF) pair as can be employed in embodiments of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable tolerance that allows a component or collection of components to function for its intended purpose as described herein.

An electrochemical-based analytical test strip for use with a hand-held test meter in the determination of an analyte (such as glucose) in a bodily fluid sample (e.g., a whole blood sample) includes an electrically insulating base layer, a patterned electrically conductive layer disposed on the electrically insulating base layer, an enzymatic reagent layer, a patterned spacer layer; and a top layer. The patterned electrically conductive layer includes at least one electrode (for example, two electrodes, namely a working electrode and a reference electrode), at least one electrode voltage sensing connection configured to sense voltage at the least one electrode (e.g., a working electrode voltage sensing connection and a reference electrode voltage sensing connection), at least one electrode track, and at least one electrode voltage sensing connection track. Moreover, the electrode voltage sensing connections are configured for operable communication of a sensed electrode voltage to an associated hand-held test meter via the at least one electrode voltage sensing connection.

Electrochemical-based analytical test strips according to embodiments of the present invention are beneficial in that the electrode voltage sensing connections and electrode voltage sensing connection tracks can be employed by an associated hand-held test meter (described herein) to measure deleterious voltage drop(s) on the electrochemical-based analytical test strip. Such measured voltage drop(s) can then be employed by the hand-held test meter to automatically (for example, dynamically) adjust a voltage bias drive(s) applied to the electrochemical-based analytical test strip by the hand-held test meter. Such automatic adjustments result in the electrode track resistance and dimensional tolerance being beneficially less critical to accurate use of embodiments of electrochemical-based analytical test strips and hand-held test meters of the present invention.

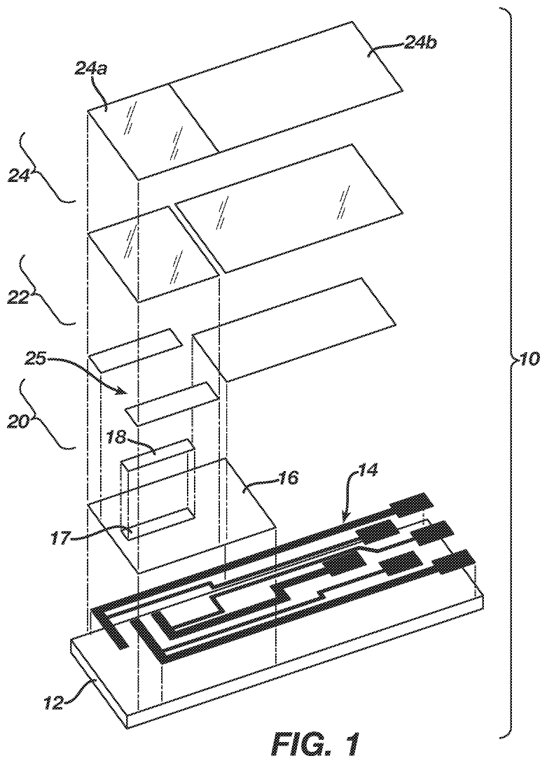

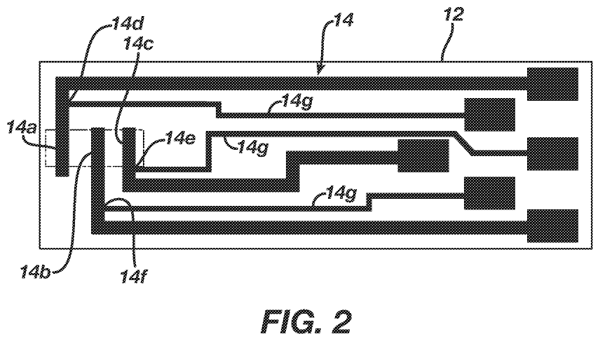

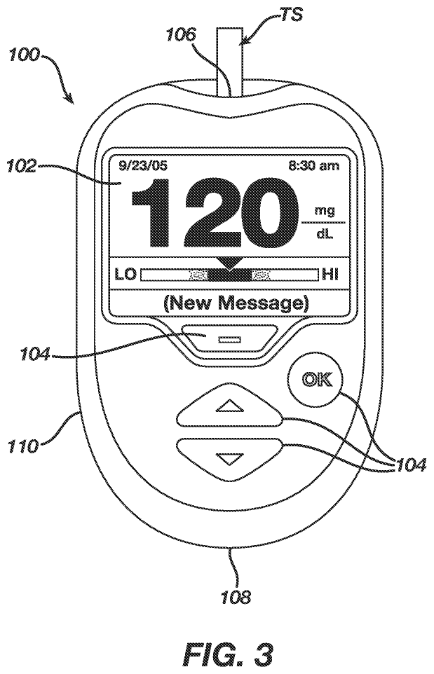

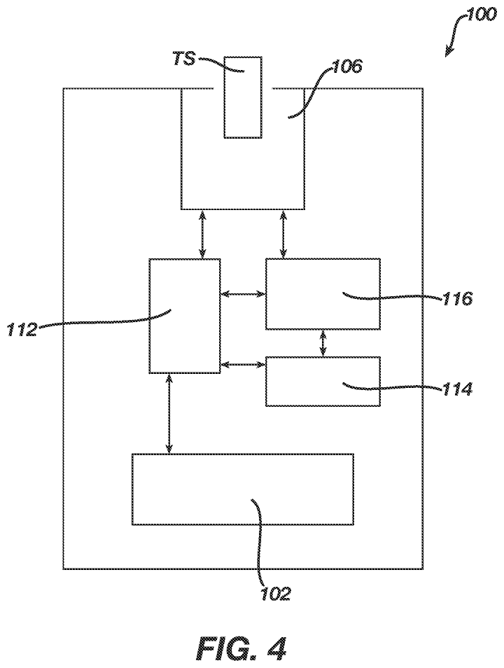

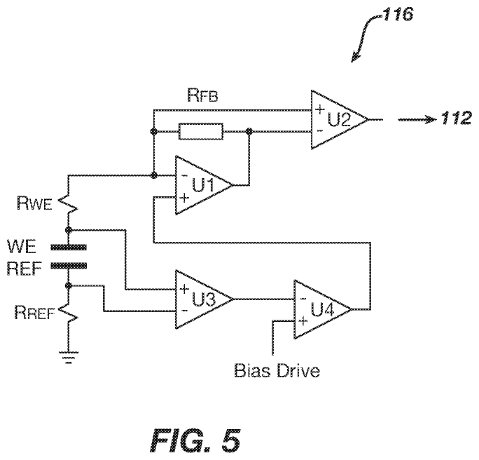

FIG. 1 is a simplified exploded perspective view of an electrochemical-based analytical test strip 10 according to an embodiment of the present invention. FIG. 2 is a simplified top view of a patterned conductor layer and substrate layer of electrochemical-based analytical test strip 10 with the location of an enzymatic reagent layer depicted by dashed lines. FIG. 3 is a simplified top view of a hand-held test meter 100 according to an embodiment of the present invention. FIG. 4 is a simplified block diagram of hand-held test meter 100. FIG. 5 is a simplified schematic diagram of an electrode bias adjustment circuit block for a single working electrode (WE) and reference electrode (REF) pair as can be employed in hand-held test meter embodiments of the present invention.

Referring to FIGS. 1 and 2, electrochemical-based analytical test strip 10 for the determination of an analyte (such as glucose) in a bodily fluid sample (for example, a whole blood sample) includes an electrically-insulating base layer 12, a patterned electrically conductive layer 14, a patterned insulation layer 16 with opening 17 therethrough, an enzymatic reagent layer 18, a patterned spacer layer 20, and a top layer consisting of a hydrophilic sub-layer 22 and a top tape 24 with portions 24a and 24b.

In the embodiment of FIGS. 1 and 2, at least the patterned spacer layer and top layer define a sample-receiving chamber 25 within electrochemical-based analytical test strip 10.

An electrochemical-based analytical test strip 10 is configured for use with a hand-held test meter (e.g., a hand-held test meter according to embodiments of the present invention and described herein) in the determination of an analyte in a bodily fluid sample, the electrochemical-based analytical test strip. See, for example, the hand-held test meter described with respect to FIGS. 3, 4 and 5.

Electrically-insulating base layer 12 can be any suitable electrically-insulating base layer known to one skilled in the art including, for example, a nylon base layer, a polycarbonate base layer, a polyimide base layer, a polyvinyl chloride base layer, a polyethylene base layer, a polypropylene base layer, a glycolated polyester (PETG) base layer, or a polyester base layer. The electrically-insulating base layer can have any suitable dimensions including, for example, a width dimension of about 5 mm, a length dimension of about 27 mm and a thickness dimension of about 0.5 mm.

Electrically-insulating base layer 12 provides structure to electrochemical-based analytical test strip 10 for ease of handling and also serves as a base for the application (e.g., printing or deposition) of subsequent layers (e.g., a patterned electrically conductor layer and an enzymatic reagent formed by ink jet printing or screen printing of an enzymatic reagent layer according to the present invention and described herein).

Patterned electrically conductive layer 14 is disposed on the electrically-insulating base layer 12 and includes a first electrode 14a, a second electrode 14b and a third electrode 14c. First electrode 14a, second electrode 14b and third electrode 14c can be, for example, configured as a counter/reference electrode, a first working electrode and a second working electrode, respectively. Therefore, the second and third electrodes are also referred to herein as working electrodes 14b and 14c and the first electrode as counter electrode 14a. Although, for the purpose of explanation only, electrochemical-based analytical test strip 10 is depicted as including a total of three electrodes, embodiments of electrochemical-based analytical test strips, including embodiments of the present invention, can include any suitable number of electrodes.

Patterned electrically conductive layer 14 also includes a first electrode voltage sensing connection 14d, a second electrode voltage sensing connection 14e and a third electrode voltage sensing connection 14f configured to sense electrode voltage at the counter/reference electrode, first working electrode and second working electrode respectively.

Patterned electrically conductive layer 14 also includes a plurality of electrode connection tracks 14g configured for operable communication of a sensed electrode voltage to a hand-held test meter.

Patterned electrically conductive layer 14 can be formed of any suitable conductive material including, for example, electrically conducting carbon-based materials including carbon inks. It should be noted that patterned electrically conductive layers employed in electrochemical-based analytical test strips according to embodiments of the present invention can take any suitable shape and be formed of any suitable materials including, for example, metal materials and conductive carbon materials.

Referring to FIGS. 1 and 2, the disposition of first electrode 14a, second electrode 14b and third electrode 14c and enzymatic reagent layer 18 are such that electrochemical-based analytical test strip 10 is configured for the electrochemical determination of an analyte (such as glucose) in a bodily fluid sample (such as a whole blood sample) that has filled sample-receiving chamber 25.

Enzymatic reagent layer 18 is disposed on at least a portion of patterned electrically conductor layer 14 (see FIG. 2 wherein the disposition of enzymatic reagent layer 18 is depicted by dashed lines). Once apprised of the present disclosure, one skilled in the art will recognize that a variety of suitable enzymatic reagents are known to one skilled in the art. Further details regarding reagent layers in general, and electrochemical-based analytical test strips in general, are in U.S. Pat. Nos. 6,241,862 and 6,733,655, the contents of which are hereby fully incorporated by reference.

Referring to FIGS. 1 and 2, patterned insulation layer 16 can be formed of any suitable electrically-insulating dielectric material including commercially available screen-printable dielectric inks.

Patterned spacer layer 20 can be formed, for example, from a screen-printable pressure sensitive adhesive commercially available from Apollo Adhesives, Tamworth, Staffordshire, UK. In the embodiment of FIG. and 2, patterned spacer layer 20 defines outer walls of the sample-receiving chamber 25. Patterned spacer layer 20 can have a thickness of, for example, approximately 110 microns, be electrically nonconductive, and be formed of a polyester material with top and bottom side acrylic-based pressure sensitive adhesive.

Top layer 24 can be, for example, a clear film with hydrophilic properties that promote wetting and filling of electrochemical-based analytical test strip 10 by a fluid sample (e.g., a whole blood sample). Such clear films are commercially available from, for example, 3M of Minneapolis, Minn. U.S.A. and Coveme (San Lazzaro di Savena, Italy). Top layer 24 can be, for example, a polyester film coated with a surfactant that provides a hydrophilic contact angle less than 10 degrees. Top layer 24 can also be a polypropylene film coated with a surfactant or other surface treatment. In such a circumstance, the surfactant coating serves as hydrophilic sub-layer 22. Top layer 24 can have a thickness, for example, of approximately 100 μm.

Electrochemical-based analytical test strip 10 can be manufactured, for example, by the sequential aligned formation of the layers depicted in FIG. 1. Any suitable techniques known to one skilled in the art can be used to accomplish such sequential aligned formation, including, for example, screen printing, ink-jet printing, photolithography, photogravure, chemical vapour deposition and tape lamination techniques. However, enzymatic reagents according to embodiments of the present invention are particularly beneficial in that they can be formulated as aqueous compositions suitable for relatively low-cost and otherwise conventional ink jet and screen printing techniques.

Electrochemically-based analytical test strip 10 is configured such that the various electrode voltages can be sensed (using the electrode's associated electrode voltage sensing connections 14d, 14e and 14f, associated electrode connection tracks 14g and an associated hand-held test meter). Using the voltages sensed at the electrodes themselves, any deleterious voltage drops can be measured and a bias drive from an associated hand-held test meter increased to compensate for the deleterious voltage drop. This can be done, for example, dynamically using a closed loop error amplifier circuit (or as elsewhere described herein).

A hand-held test meter for use with an electro-chemical-based analytical test strip in the determination of an analyte in a bodily fluid sample according to embodiments of the present invention includes a housing, a strip port connector disposed at least partially within the housing and configured to receive an electro-chemical based analytical test strip, a micro-controller disposed in the housing and configured to generate a micro-controller command signal, an electrode bias drive circuit block disposed in the housing and configured to generate a bias drive signal based on the micro-controller command signal, and an automatic bias drive adjustment circuit block disposed in the housing and configured to receive at least one sensed electrode voltage and to adjust a bias drive signal from the electrode bias drive circuit block based on the least one sensed electrode voltage to create an adjusted bias drive signal.

Referring to FIGS. 3, 4 and 5, hand-held test meter 100 includes a display 102, a plurality of user interface buttons 104, a strip port connector 106, a USB interface 108, and a housing 110 (see FIG. 1). Referring to FIG. 2 in particular, hand-held test meter 100 also includes a micro-controller block 112, an electrode bias drive circuit block 114 disposed in the housing and configured to generate a bias drive signal based on the micro-controller command signal, and an automatic bias drive adjustment circuit block 116 disposed in the housing and configured to receive at least one sensed electrode voltage and to adjust a bias drive signal from the electrode bias drive circuit block based on the least one sensed electrode voltage to create an adjusted bias drive signal, and other electronic components (not shown in the FIGs.) for applying an electrical bias (e.g., an alternating current [AC] and/or direct current [DC] bias) to an electrochemical-based analytical test strip (labeled TS in FIGS. 3 and 4), and also for measuring an electrochemical response (e.g., plurality of test current values, phase, and/or magnitude) and determining an analyte or characteristic based on the electrochemical response. To simplify the current descriptions, the figures do not depict all such electronic circuitry.

Display 102 can be, for example, a liquid crystal display or a bi-stable display configured to show a screen image. An example of a screen image during the determination of an analyte in a bodily fluid sample may include a glucose concentration, a date and time, an error message, and a user interface for instructing a user how to perform a test. Examples of screen images during use of the operating range test strip simulation circuit block may be an image reporting that a hand-held test meter operating range test passed, or an image reporting that the hand-held test meter operating range test has resulted in an error.

Strip port connector 106 is configured to operatively interface with an electrochemical-based analytical test strip TS, such as an electrochemical-based analytical test strip configured for the determination of hematocrit and/or glucose in a whole blood sample. Therefore, the electrochemical-based analytical test strip is configured for operative insertion into strip port connector 106 and to operatively interface with micro-controller block 112 via, for example, suitable electrical contacts, wires, electrical interconnects or other structures known to one skilled in the art.

USB Interface 108 can be any suitable interface known to one skilled in the art. USB Interface 108 is an electrical component that is configured to power and provide a data line to hand-held test meter 100.

Micro-controller block 112 also includes a memory sub-block that stores suitable algorithms for the determination of an analyte based on the electrochemical response of an analytical test strip and to also determine a characteristic (e.g., hematocrit) of the introduced bodily fluid sample. Micro-controller block 112 is disposed within housing 110 and can include any suitable micro-controller and/or micro-processer known to those skilled in the art. Suitable micro-controllers include, but are not limited to, micro-controllers available commercially from Texas Instruments (Dallas, Tex., USA) under the MSP430 series of part numbers; from ST MicroElectronics (Geneva, Switzerland) under the STM32F and STM32L series of part numbers; and Atmel Corporation (San Jose, Calif., USA) under the SAM4L series of part numbers).

Referring, in particular, to FIG. 5, an exemplary, but non-limiting, automatic bias drive adjustment circuit block 116 for a single working electrode and associated counter/reference electrode is depicted using conventional electronic component symbols. In FIG. 5, WE is a working electrode, REF is a counter/reference electrode, R represents a resistor, and U represents an amplifier. Automatic bias drive adjustment circuit block 116 can be generally considered a closed loop error amplifier circuit.

The operation of hand-held test meter 100 is now described with reference to FIGS. 3, 4 and, in particular, 5. During operation, system micro-controller 112 commands a required bias onto the bias drive signal (see FIG. 5), via an electrode bias drive circuit block 114 (for example, via a DAC serving as an electrode bias drive circuit block). R_{we} and R_{ref} represent the additional resistance in the circuit introduced by the electrode tracks. U3 is a difference amplifier with a gain of ×1 that provides a measurement of the actual voltage across an electrode WE. U4 is an error amplifier that compares the measured electrode voltage against the commanded bias drive value. This error amplifier U4 typically has a high gain (at least 100).

U1 is, for example, a trans-impedance amplifier that uses U4 output to both drive a bias across the electrodes (WE and REF in FIG. 5), and to measure a working electrode WE current. The U4 drive is effectively slightly above the required bias drive, compensating for the voltage drop across the electrode track resistance. All the current flowing through the working electrode WE is also pushed through R_{FB}, and therefore an accurate measurement of the voltage across R_{FB} provides an indication of working electrode current. Depending upon the maximum working electrode current, R_{FB} is scaled to provide maximum ADC input for maximum WE current. The value R_{FB} be, for example, approximately around 100K ohm for a patterned electrically conductive layer formed of carbon. U2 is a difference amplifier with a gain of ×1, and provides an accurate measurement of the voltage across R_{FB} to the output of U1. U1 output then feeds into an ADC input of the system microcontroller.

Once apprised of the present disclosure, one skilled in the art will recognize that automatic bias drive circuit blocks employed in hand-held test meters according to embodiments of the present invention can take various forms and are not limited to the embodiment depicted in FIG. 5.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and compositions of matter within the scope of these claims and their equivalents be covered thereby.

We claim:

1. A hand-held test meter for use with an electrochemical-based analytical test strip in the determination of an analyte in a bodily fluid sample, the hand-held test meter comprising:
a housing;
a strip port connector disposed at least partially within the housing and configured to receive an electro-chemical based analytical test strip;
a micro-controller disposed in the housing and configured to generate a micro-controller command signal, the micro-controller comprising an analog to digital signal converter input;
an electrode bias drive circuit block disposed in the housing and configured to generate a bias drive signal based on the micro-controller command signal; and
an automatic bias drive adjustment circuit block disposed in the housing and configured to receive at least one sensed electrode voltage and to adjust a bias drive signal from the electrode bias drive circuit block based on the least one sensed electrode voltage to create an adjusted bias drive signal, wherein the automatic bias drive adjustment circuit block comprises:
a first difference amplifier configured to provide the at least one sensed electrode voltage;
an error amplifier configured to compare the at least one sensed electrode voltage to the generated bias drive signal;
a trans-impedance amplifier configured to use the output of the error amplifier to both drive the adjusted bias drive signal and to measure at least one sensed electrode current;
a resistor across the trans-impedance amplifier; and
a second difference amplifier configured to provide a measurement of the voltage across the resistor to the output of the trans-impedance amplifier, wherein the trans-impedance amplifier is further configured to feed into the analog to digital converter input of the micro-controller.

2. The hand-held test meter according to claim 1, wherein the electrode bias circuit block is an analog to digital converter (DAC).

3. The hand-held test meter according to claim 1, wherein the hand-held test meter is configured for the determination of glucose in a whole blood sample applied to the electrochemical-based analytical test strip.

4. An electrochemical-based analytical test strip for use with the hand-held test meter of claim 1, in the determination of an analyte in a bodily-fluid sample, the electrochemical-based analytical test strip comprising:
an electrically insulating base layer;
a patterned electrically conductive layer disposed on the electrically insulating base layer and including at least one electrode;
an enzymatic reagent layer disposed on the at least one electrode;
a patterned spacer layer; and
a top layer, wherein the patterned electrically conductive layer includes:
a plurality of electrodes;
at least one voltage electrode sensing connection configured to sense electrode voltage of at least one of the plurality of electrodes;
a plurality of electrode tracks; and
at least one electrode voltage sensing connection track configured for operable communication of a sensed electrode voltage to the hand-held test meter.

5. The electrochemical-based analytical test strip according to claim 4, wherein the plurality of electrodes includes:
a first working electrode;
a second working electrode; and
a reference electrode.

6. The electrochemical-based analytical test strip according to claim 5, wherein the at least one electrode voltage sensing connection includes:
a first electrode voltage sensing connection in electrical connection with the first working electrode;
a second electrode voltage sensing connection in electrical connection with the second working electrode; and
a third electrode voltage sensing connection in electrical connection with the reference electrode.

7. The electrochemical-based analytical test strip according to claim 4, wherein the patterned electrically conductive layer comprises a carbon-containing material.

8. The electrochemical-based analytical test strip according to claim 4, wherein each of the plurality of voltage sensing connections are electrically connected to a single one of the electrodes at a distance of less than 10 mm.

9. The electrochemical-based analytical test strip according to claim 4, wherein each of the plurality of voltage sensing connections includes:
an electrode voltage sensing track; and
an electrode voltage sensing pad.

10. The electrochemical-based analytical test strip according to claim 9, wherein a voltage drop of the electrode voltage sensing track is insignificant in comparison to a voltage drop at the associated electrode.

11. In combination, a hand-held test meter for use with an electrochemical-based analytical test strip in the determination of an analyte in a bodily fluid sample, in which the hand-held test meter comprises:
a housing;
a strip port connector disposed at least partially within the housing and configured to receive an electro-chemical based analytical test strip;
a micro-controller disposed in the housing and configured to generate a micro-controller command signal, the micro-controller comprising an analog to digital signal converter input;
an electrode bias drive circuit block disposed in the housing and configured to generate a bias drive signal based on the micro-controller command signal; and
an automatic bias drive adjustment circuit block disposed in the housing and configured to receive at least one sensed electrode voltage and to adjust a bias drive signal from the electrode bias drive circuit block based on the least one sensed electrode voltage to create an adjusted bias drive signal, wherein the automatic bias drive adjustment circuit block comprises:
a first difference amplifier configured to provide the at least one sensed electrode voltage;
an error amplifier configured to compare the at least one sensed electrode voltage to the generated bias drive signal;
a trans-impedance amplifier configured to use the output of the error amplifier to both drive the adjusted bias drive signal and to measure at least one sensed electrode current;
a resistor across the trans-impedance amplifier; and
a second difference amplifier configured to provide a measurement of the voltage across the resistor to the output of the trans-impedance amplifier, wherein the trans-impedance amplifier is further configured to feed into the analog to digital converter input of the micro-controller.

12. The combination according to claim 11, wherein the electrode bias circuit block is an analog to digital converter (DAC).

13. The combination according to claim 11, wherein the hand-held test meter is configured for the determination of glucose in a whole blood sample applied to the electrochemical-based analytical test strip.

14. The combination according to claim 11, wherein the electrochemical-based analytical test strip comprises:
an electrically insulating base layer;
a patterned electrically conductive layer disposed on the electrically insulating base layer and including at least one electrode;
an enzymatic reagent layer disposed on the at least one electrode;
a patterned spacer layer; and
a top layer, wherein the patterned electrically conductive layer includes:
a plurality of electrodes;
at least one voltage electrode sensing connection configured to sense electrode voltage of at least one of the plurality of electrodes;
a plurality of electrode tracks; and
at least one electrode voltage sensing connection track configured for operable communication of a sensed electrode voltage to the hand-held test meter.

15. The combination according to claim 14, wherein the plurality of electrodes includes:
a first working electrode;
a second working electrode; and
a reference electrode.

16. The combination according to claim 15, wherein the at least one electrode voltage sensing connection includes:
a first electrode voltage sensing connection in electrical connection with the first working electrode;
a second electrode voltage sensing connection in electrical connection with the second working electrode; and
a third electrode voltage sensing connection in electrical connection with the reference electrode.

17. The combination according to claim 14, wherein the patterned electrically conductive layer comprises a carbon-containing material.

18. The combination according to claim 14, wherein each of the plurality of voltage sensing connections are electrically connected to a single one of the electrodes at a distance of less than 10 mm.

19. The combination according to claim 14, wherein each of the plurality of voltage sensing connections includes:
an electrode voltage sensing track; and
an electrode voltage sensing pad.

20. The combination according to claim 19, wherein a voltage drop of the electrode voltage sensing track is insignificant in comparison to a voltage drop at the associated electrode.
